(19) United States
(12) Patent Application Publication
(10) Pub. No.: US 2026/0224442 A1
(43) Pub. Date: Aug. 6, 2026

(54) INDICATING BAND AND METHOD OF INDICATING INFORMATION USING THE SAME

(71) Applicant: Shaya Naimi, Pasadena, CA (US)

(72) Inventor: Shaya Naimi, Pasadena, CA (US)

(21) Appl. No.: 19/531,340

(22) Filed: Feb. 5, 2026

Related U.S. Application Data
(60) Provisional application No. 63/754,234, filed on Feb. 5, 2025.

(51) Int. Cl. A61J 1/20; A61F 9/08

(52) U.S. Cl. CPC A61J 1/2003; A61F 9/08; A61J 2205/40

(57) ABSTRACT

An indicating band configured to be removably attached to a plurality of differently shaped containers, and having improved dimensions and/or improved composition is provided. The band includes a body comprising a flexible material; and a protrusion on an outer surface of the body and extending away from the body. The protrusion may include a first protrusion and a second protrusion, the first protrusion having a shape and/or color different from that of the second protrusion.

### CROSS-REFERENCE TO RELATED APPLICATIONS

This application claims priority to and the benefit of U.S. Provisional Application No. 63/754,234, filed on February 5, 2025, the entire content of which is incorporated herein by reference.

### BACKGROUND

### Field

Aspects of embodiments of the present disclosure relate generally to an indicating band and a method of indicating information using the indicating band.

### Description of the Related Art

Individuals with impaired vision, or those without vision altogether, have difficulty independently interacting with and/or differentiating between small containers in the environment as many containers share a similar form factor. In particular, visually impaired individuals have difficulty in discerning information (e.g., dosage instructions, serving suggestions, etc.) written on the small containers. This issue arises in the context of relatively benign areas, such as spices for cooking, and critical areas, such as medications. Many individuals with limited or no vision are self-sufficient except for these areas simply due to manufacturers using containers with non-descriptive or similar form factors or usage of generic prescription containers for medications. Thus, some products for helping these individuals identify different containers and information associated therewith by touch (e.g., tactile sensations) have been developed. However, these devices have limited applications.

Presently, devices for assisting a user with applying and/or dispensing specific quantities of medication from a container have limited functionality. For example, current devices are made from rigid materials (e.g., stiff plastics, metals, or the like). Thus, a device must be designed for a specific container shape and size and cannot be attached to containers having different shapes. In other words, the device can only be attached to a bottle having specific dimensions and attempting to use the device with smaller or larger containers will cause the device to fall off or not fit, respectively. This requires that a user, such as an individual that requires medication from differently sized containers, to purchase multiple devices for use with the differently sized containers, leading to reduced functionality and increased cost of use.

Additionally, current devices may be unintentionally displaced from the container due to the device having a rigid structure causing less friction to exist between the device and the container. This may further frustrate a user’s experience when using the device, especially if the user has any physical limitations (e.g., limited dexterity). Repeated use may simply cause the device to fall off the container to which it is attached, or worse, while the user is administering a medication from the container.

Existing devices use hard plastic protrusions positioned near the neck of the container, which increases the risk of injury during medication administration, particularly around sensitive areas, such as the eye in the case of eye drops.

### SUMMARY

A need exists for an improved indicating band having improved dimensions and/or composition to provide enhanced functionality, improved handling, and safer application when attached to one or more containers.

Embodiments of the present disclosure provide an indicating band formed of (or made of) materials and/or dimensioned to provide optimal use and safety to users.

Embodiments of the present disclosure relate to an indicating band (hereinafter referred to as a “band”) that is configured to be removably attached to a plurality of containers having different external shapes. The band may include a body including a flexible material, and a protrusion on an outer surface of the body and extending away from the body. The protrusion may include a first protrusion and a second protrusion, and the first protrusion may have a shape, size, color, softness, surface treatment (e.g., roughness), and/or other physical characteristic(s) that is different from that of the second protrusion.

However, embodiments of the present disclosure are not limited to those set forth herein. The above and other embodiments of the present disclosure (and aspects and features thereof) will become more apparent to one of ordinary skill in the art to which the present disclosure pertains by referencing the detailed description of the present disclosure below. For example, although embodiments of the present disclosure are described in connection with a person interacting with medicine containers (e.g., prescription bottles), embodiments of the present disclosure are not limited thereto. For example, embodiments of the present disclosure are convenient for any situation where containers need to be quickly distinguished without relying on vision, such as busy kitchens, containers stored in hard to reach or see areas, etc.

According to an embodiment of the present disclosure, a band is configured to be removably attached to a plurality of containers having different external shapes, respectively, and the indicating band includes a body having a closed loop shape and comprising a flexible material; and a protrusion on an outer surface of the body and extending away from the body.

In an embodiment, the body may include at least one of rubber, silicone, or polyurethane.

In an embodiment, the protrusion may be integrally formed with the body.

In an embodiment, the protrusion may be removably coupled to the body.

In an embodiment, the protrusion may be removably coupled to the body by at least one of a magnetic coupling, a screw coupling, or a hook-and-loop coupling.

In an embodiment, the protrusion may have at least one of a square shape, a rectangle shape, a triangle shape, a semi-triangle shape, semi-oval shape, or a semi-circular shape in a plan view.

In an embodiment, the protrusion may be a first protrusion, wherein the band may further include a second protrusion on the outer surface of the body, extending away from the body, and spaced apart from the first protrusion, and wherein the first protrusion may have a first shape, and the second protrusion may have a second shape that is different than the first shape.

In an embodiment, the protrusion may be a first protrusion, wherein the indicating band may further include a second protrusion on the outer surface of the body, extending away from the body, and spaced apart from the first protrusion, and wherein the first protrusion may have a first color, and the second protrusion has a second color that is different than the first color.

In an embodiment, the protrusion may have a width decreasing in a direction away from the body.

In an embodiment, the protrusion may have a height less than or equal to 3.0 millimeters.

In an embodiment, the protrusion may have a width less than or equal to a width of the body.

According to another embodiment of the present disclosure, a system includes a plurality of containers having different external shapes; and a flexible indicating band configured to be individually, removably attached to respective ones of the plurality of containers, the indicating band including a body having a closed-loop shape and configured to individually extend around each of the plurality of containers; and a plurality of protrusions on the outer surface of the body and extending away from the body, a first protrusion from among the plurality of protrusions having a shape different from that of a second protrusion from among the plurality of protrusions.

In an embodiment, the body may be configured to extend around a first portion of at least one of the plurality of containers and around a second portion of the at least one of the plurality of containers, and wherein the first and second portions of the at least one of the containers may be spaced from each other and may have different circumferences with respect to each other.

In an embodiment, the protrusion may be removably coupled to the body.

In an embodiment, the first protrusion may have a shape or color different from that of the second protrusion, the shape or the color corresponding to a dosage of contents of the at least one of the plurality of containers.

According to another embodiment of the present disclosure, a method for indicating information associated with contents of a container to a user via tactile features on a flexible band, the method including providing a plurality of flexible bands, respective ones of the plurality of flexible bands being configured to be removably attached around the container, and having a plurality of tactile protrusions; associating one or more characteristics of the contents of the container with a tactile pattern formed by the plurality of tactile protrusions of the respective ones of the plurality of flexible bands, the tactile patterns of the plurality of flexible bands configured to be perceived by touch, and the characteristics including at least one of: contents of the container; a dosage or usage amount; or a usage instruction; attaching the plurality of flexible bands to the container, the flexible bands indicating the one or more characteristics of the contents of the container; and enabling the user to identify the associated characteristics of the contents of the container by tactilely perceiving the plurality of protrusions on each of the plurality of flexible bands without reliance on visual information.

In an embodiment, the tactile patterns of the respective ones of the plurality of flexible bands may represent a same characteristic for a plurality of containers.

In an embodiment, the method for indicating information may further include modifying the tactile patterns of the respective ones of the plurality of flexible bands to represent a different characteristic.

In an embodiment, the method for indicating information may further include removing at least one of the plurality of flexible bands from a first container and attaching the at least one of the plurality of flexible bands to a second container; and modifying the tactile pattern formed by the plurality of protrusions of the at least one of the plurality of flexible bands to represent the different characteristic, the different characteristic being of contents of the second container.

In an embodiment, different subsets or positions of the plurality of protrusions of at least one of the plurality of flexible bands may represent a different characteristic.

### BRIEF DESCRIPTION OF THE DRAWINGS

The above and other aspects and features of the present disclosure will become more apparent by describing, in detail, embodiments thereof with reference to the attached drawings, in which:

FIG. 1 is a perspective view of an indicating band according to embodiments of the present disclosure.

FIG. 2 is a plan view of the indicating band of FIG. 1 according to embodiments of the present disclosure.

FIG. 3 is a plan view of the indicating band of FIG. 1 according to embodiments of the present disclosure.

FIG. 4 is a perspective view of an indicating band attached to different containers according to embodiments of the present disclosure.

FIG. 5 is a perspective view of an indicating band according to embodiments of the present disclosure.

FIG. 6 is a plan view of the indicating band of FIG. 5 according to embodiments of the present disclosure.

FIGS. 7-12 is a plan view of a plurality of protrusions according to embodiments of the present disclosure.

### DETAILED DESCRIPTION

The present disclosure will now be described more fully hereinafter with reference to the accompanying drawings, in which embodiments of the present disclosure are shown. The present disclosure may, however, be embodied in different forms and should not be construed as limited to the embodiments set forth herein. Rather, these embodiments are provided so that this disclosure will be thorough and complete and will fully convey the scope of the present disclosure to those skilled in the art.

In the figures, dimensions of the various elements, layers, etc. may be exaggerated for clarity of illustration. The same reference numerals designate the same elements. As used herein, the term “and/or” includes any and all combinations of one or more of the associated listed items. Further, the use of "may" when describing embodiments of the present invention relates to "one or more embodiments of the present invention." Expressions, such as “at least one of,” when preceding a list of elements, modify the entire list of elements and do not modify the individual elements of the list. Also, the term “exemplary” is intended to refer to an example or illustration. As used herein, the terms “use,” “using,” and “used” may be considered synonymous with the terms “utilize,” “utilizing,” and “utilized,” respectively. As used herein, the terms "substantially," "about," and similar terms are used as terms of approximation and not as terms of degree, and are intended to account for the inherent variations in measured or calculated values that would be recognized by those of ordinary skill in the art.

It will be understood that, although the terms “first,” “second,” etc. may be used herein to describe various elements, these elements should not be limited by these terms. These terms are only used to distinguish one element from another element. For instance, a first element discussed below could be termed a second element without departing from the teachings of the present disclosure. Similarly, the second element could also be termed the first element.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” or "over" the other elements or features. Thus, the term “below” may encompass both an orientation of above and below. The device may be otherwise oriented (rotated 90 degrees or at other orientations), and the spatially relative descriptors used herein should be interpreted accordingly.

The terminology used herein is for the purpose of describing particular example embodiments of the present invention and is not intended to be limiting of the described example embodiments of the present invention. As used herein, the singular forms “a” and “an” are intended to include the plural forms as well, unless the context clearly indicates otherwise. It will be further understood that the terms "includes," "including," “comprises,” and/or “comprising,” when used in this specification, specify the presence of stated features, integers, steps, operations, elements, and/or components but do not preclude the presence or addition of one or more other features, integers, steps, operations, elements, components, and/or groups thereof.

Embodiments of the present disclosure will hereinafter be described with reference to the accompanying drawings.

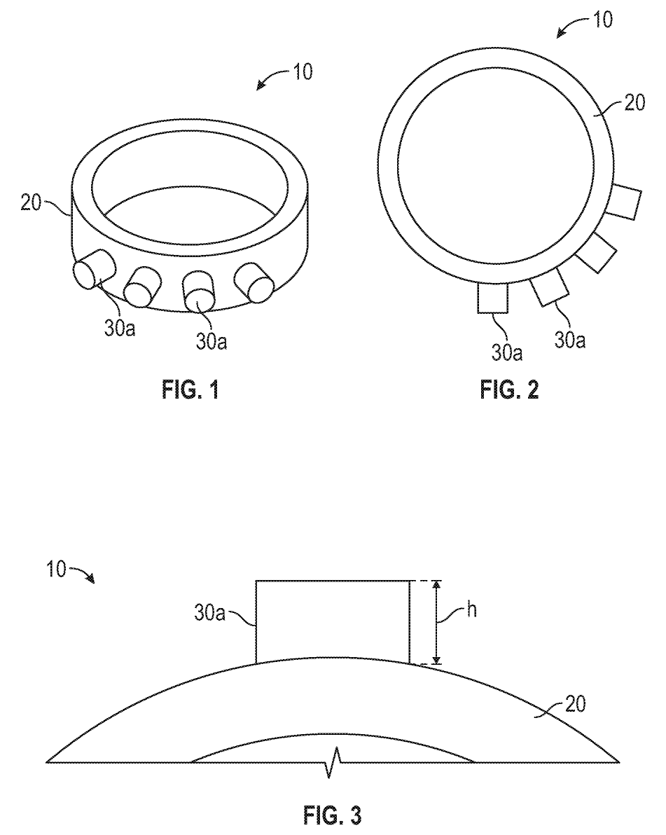

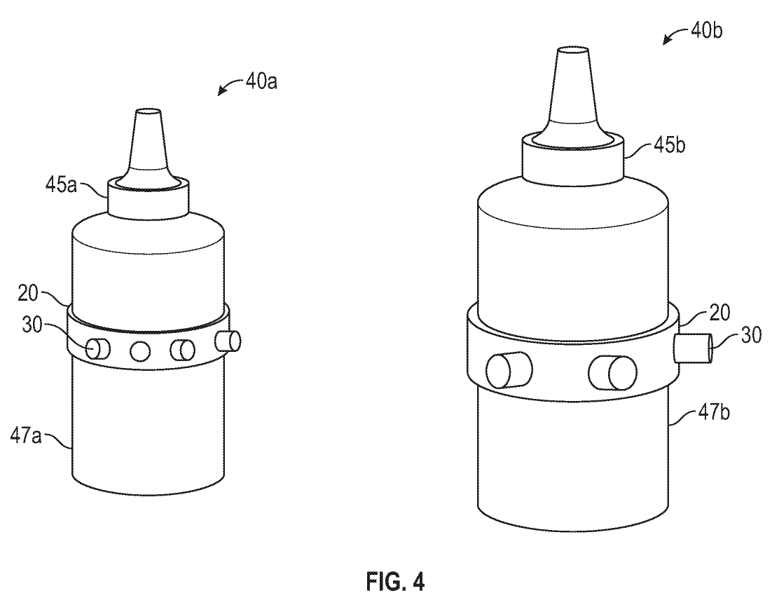

FIGS. 1, 2, and 3 show a band according to some embodiments of the present disclosure. FIG. 4 shows a band attached to different containers according to some embodiments of the present disclosure.

According to some embodiments of the present disclosure, a band 10 includes a body 20 having a closed loop shape and a plurality of protrusions 30 (e.g., protrusions 30a in FIG. 1) on a surface (e.g., an outer surface) of the body 20. The protrusions 30 may extend away from the body 20. In some embodiments, the protrusions 30a may respectively extend away from the body 20 in a direction normal to the outer surface of the body 20. In some embodiments, such as is shown in FIG. 1, a first protrusion 30-1a from among the plurality of protrusions 30a may extend in a first direction D1 away from the body 20 and a second protrusion 30-2a from among the plurality of protrusions 30a may extend in a second direction D2 away from the body 20. The first direction D1 may be different from (e.g., cross) the second direction D2. In some embodiments, the first direction D1 and the second direction D2 may each be normal to the band 20 at the area where the respective protrusion 30a extends from.

In one or more embodiments, the band 10 includes (e.g., is formed of) a flexible (e.g., elastic) and/or frictional material, for example, at least one of rubber, silicone, polyurethane, and/or the like. By using a soft, flexible material, impact between the band 10 and the user will not result in injury, for example, with the band 10 is attached to a eye dropper that will be near the user’s eye. In some embodiments, the protrusions 30 may be formed of a flexible material, for example, the same flexible materials as the body 20. However, in some embodiments, the protrusions 30 may be formed of a material different from that of the body 20. The band 10 may be placed on any portion of a container. In some embodiments, such as is shown in FIG. 4, the band 10 may be attached to a container 40a at (e.g., around) a neck portion 45a or a body portion 47a thereof, even if a width of the neck portion 45a is less than that of the body portion 47a because the body 20 includes a flexible material that can stretch to fit various sized parts of the container 40a. The flexible nature of the body 20 allows the band 10 to stretch to fit around the body portion 47a of the container 40a, which has a greater circumference than the neck portion 47a. The user is able to attach the band 10 to the neck portion 45a of the container 40a, which has a small circumference than the body portion 47a, after the band 10 has been attached to the body portion 47a due to the elastic nature of the body 20 allowing the band 10 to shrink from an expanded state to extend around the neck portion 45a while maintaining sufficient friction with the neck portion 45a to avoid being unintentionally disconnected therefrom. Similarly, the band 10 may be detached from the container 40a and attached to a second container 40b at the body portion 47b or at the neck portion 45b thereof.

In one or more embodiments, the band 10 may be configured to be removably attached to a plurality of individual (e.g., separate) containers having different external shapes. In some embodiments, when the first container has one or more dimensions different from that of the second container, the band 10 attached to the first container may be removed and attached to the second container despite a different in dimensions. Despite the first and second containers having different dimensions, the flexible nature of the body 20 allows the band 10 to stretch to extend around the second containers having a greater circumference. In some embodiments, such as is shown in FIG. 4, the band 10 may be configured to be removably attached to the first container 40a and the second container 40b. The first container 40a has a different external shape than the second container 40b; for example, the second container 40b may have a greater external circumference than that of the first container 40a. The flexible nature of the band 10 allows it to be attached to any portions (e.g., 45a, 47a, 45b, or 47b) of either of the first container 40a or the second container 40b.

Additionally, the frictional nature of the body 20 may cause an increased frictional force between an inner surface of the body 20 and a side surface of each of the containers, thereby reducing a risk of the band 10 unintentionally detaching from any of the containers.

In one or more embodiments, the band 10 includes one or more protrusions 30 on an outer surface of the body 20. The quantity of the one or more protrusions 30 may be based on a value to be indicated by the band 10, for example, a value corresponding to at least one of the plurality of containers. In some embodiments, such as is shown in FIG. 1, when a dosage of medication or the like in a container is to be taken four times over a certain time period (e.g., a day, a week, etc.), a band 10 having four protrusions 30a may be placed around the container. Thus, the user can rely on the four protrusions 30a to indicate the frequency with which the medication is to be consumed even when the user has limited vision. It is understood that one skilled in the art may select any suitable number of protrusions within the spirit of the present disclosure and that the number of protrusions may indicate any relevant specification of what is stored in the container, such as the number of pills to be consumed daily, an amount of medication to be consumed, etc. It is also contemplated that a user may use multiple different bands 10 on the same container, with each band 10 indicating a different characteristic. For example, a user may place a first band around a neck of the container to indicate a frequency at which the medication is to be consumed and may place a second band around a body of the same container to indicate a number or amount of consumed at each dose.

In one or more embodiments, the protrusions 30 may be integrally formed with the body 20. In some embodiments, the protrusions 30 may be part of (e.g., attached) the body 20. By being integrally formed, the protrusions 30 may have improved structural integrity, which can improve lifespan when the protrusions 30 are physically manipulated (e.g., by a user’s finger) in a repeated manner.

In one or more embodiments, the protrusions 30 may be removably coupled to the body 20 through a coupling mechanism. In some embodiments, any of the protrusions 30 may be removably coupled to the body 20 by at least one of a magnetic coupling, a screw coupling, a Velcro^{®} (a registered trademark owned by VELCRO INDUSTRIES, B.V.) coupling (also known as a hook-and-loop fastener), and/or the like. By being removably coupled to the body 20, the protrusions 30 may be changed (e.g., replaced) such that a same band may include various different protrusions thereon having different shapes, sizes, tactile feel (e.g., roughness), and/or colors. Because different protrusions may be included on (e.g., attached to) the same band, the same band may be configured to be removably attached to a plurality of containers having different values (e.g., dosages) associated therewith or may be used to indicate multiple different characteristics of a medication (e.g., dosage and frequency), and the functionality of the band may be enhanced.

In one or more embodiments, the protrusions 30b may have a width less than of the body 20, the width extending in a direction perpendicular to an extension direction of the height of the protrusions 30b. In some embodiments, such as is shown in FIG. 3, the width of a protrusion 30-1b of the protrusions 30b may be less than the width of the body 20, and an edge of a bottom surface of the protrusion 30-1b may be spaced apart from an edge of the body 20.

In one or more embodiments, the protrusions 30 may have a height less than or equal to about 3.0 millimeters. In some embodiments, such as is shown in FIG. 3, the protrusions 30a may have a height equal to about 3.0 millimeters. By having a height within a range of less than or equal to 3.0 millimeters, a likelihood of the protrusions 30 detaching and/or breaking during a use of the band 10 may decrease.

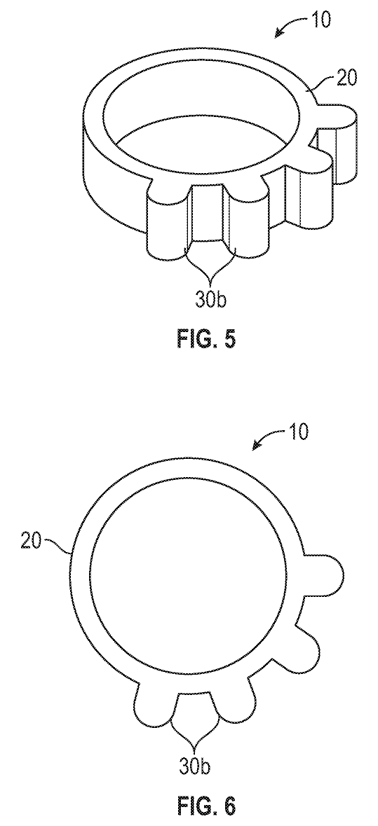

FIGS. 5 and 6 show a band according to some embodiments of the present disclosure.

In one or more embodiments, the protrusions 30b may have a width equal to that of the body 20. In some embodiments, such as is shown in FIG. 3, the width of each of the protrusions 30b may be the same as the width of the body 20. In this way, the protrusions 30b may have an increased surface area contacting the body 20, thereby improving structural integrity and reducing the likelihood of the protrusions 30b becoming defective, for example, separating from the body 20.

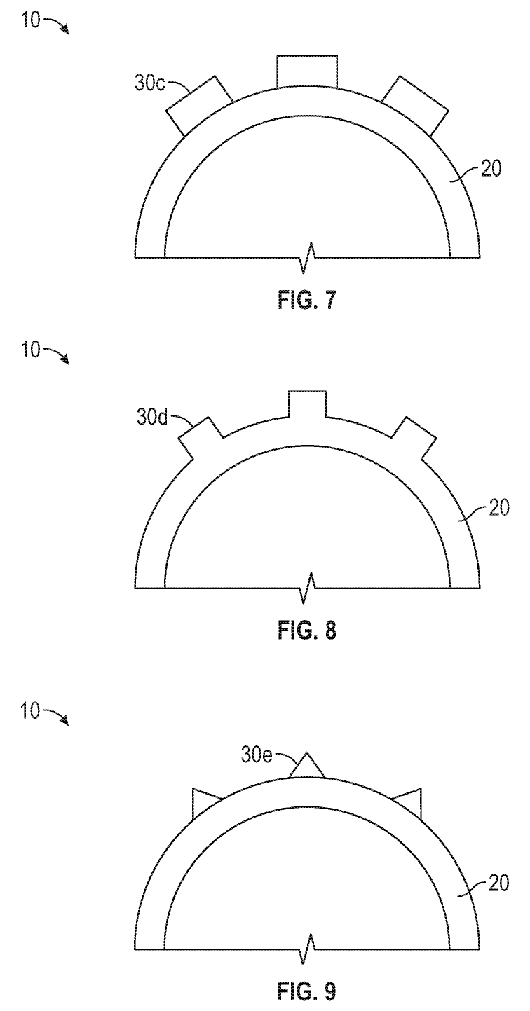

FIGS. 7-12 show a plurality of protrusions according to embodiments of the present disclosure.

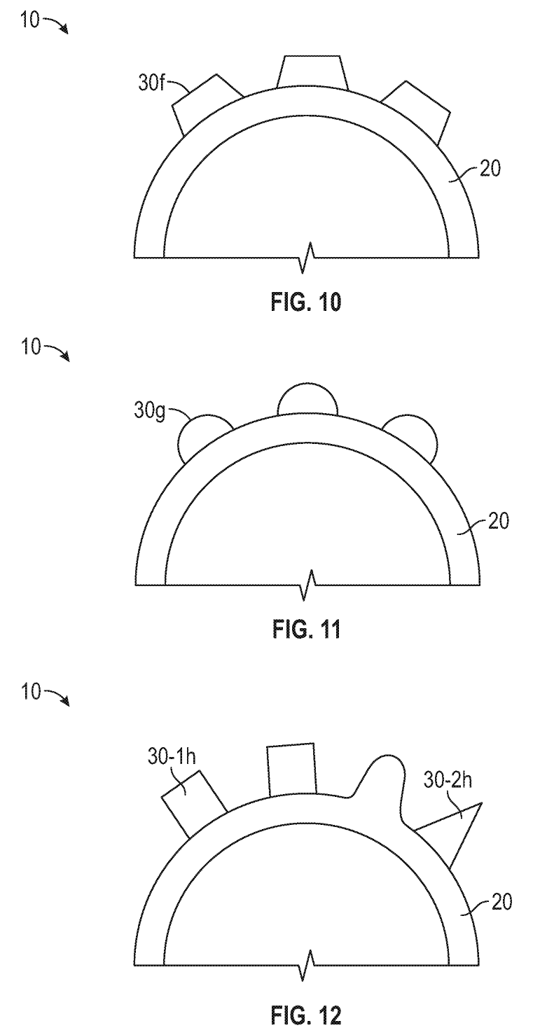

In one or more embodiments, the protrusions 30 may have at least one of a square shape (see, e.g., FIGS. 1 and 8), a rectangle shape (see, e.g., FIG. 7), a triangle shape (see, e.g., FIG. 9), a semi-triangle shape (see, e.g., FIG. 10), a semi-circular shape (see, e.g., FIG. 11), a semi-oval shape (see, e.g., FIG. 5), and/or the like, in a plan view. In some embodiments, a width of a protrusion from among the protrusions 30 may decrease in a direction away from the body 20, thereby reducing the deformation caused due to repetitive physical manipulation of the protrusions 30 by a user. It is understood that one skilled in the art may select any suitable shape for the protrusions 30 within the spirit of the present disclosure. By including protrusions having a particular or different shapes on the body of the band, the ease with which a user may identify the value associated with the container, for example, a dosage, may be improved. In some cases, a user may associate a type and/or the like of a medication in the container with a particular shape of a protrusion of the plurality of protrusions.

In one or more embodiments, a shape of a first protrusion of the protrusions 30 may be different from a shape of a second protrusion. For example, such as is shown in FIG. 12, the first protrusion 30-1h may have a rectangle shape, and the second protrusion 30-2h may have a triangular shape, and the rectangle and triangle shapes may correspond to one or more of the plurality of containers.

In one or more embodiments, a color of a first protrusion from among the protrusions 30 may be different from a color of a second protrusion. For example, the color of a first protrusion 30-1h may be a green color, and the color of a second protrusion 30-2h may be a red color, and the green and red colors may correspond to different values corresponding to the plurality of containers.

When the band 10 includes protrusions 30 having different shapes, colors and/or other characteristics, the band 10 may be attached to one or more containers to indicate a plurality of values corresponding to the containers, for example, a dosage and a type of a medicine and/or the like to be dispensed from the containers. In this way, the functionality of the band 10 is improved and broadened.

In one or more embodiments, a method for indicating information associated with contents of a container to a user via tactile features on a flexible band may include a first step S10 of providing a plurality of flexible indicating bands configured to be removably attached around the container, and having a plurality of tactile protrusions. In some embodiments, a single protrusion may be included on the body, while in other embodiments, two or more protrusions may be included. In some embodiments, the plurality of flexible indicating bands may have a body to which the plurality of tactile protrusions may be attached, for example on the outer surface of the body. The bands may be disposed around the container, and may be adjacent to each other in an extension direction of the container.

In one or more embodiments, the indicating of information includes a second step S20 of associating one or more characteristics of the contents of the container with a tactile pattern formed by the plurality of tactile protrusions of respective ones of the bands, the tactile patterns configured to be perceived by touch. The tactile patterns may be formed by assigning a particular quantity, shape and/or colors to the plurality of protrusions. In some embodiments, the characteristics may include at least one of contents of the container; a dosage or usage amount; or a usage instruction.

In one or more embodiments, the indicating of information includes a third step S30 of attaching the plurality of bands to the container. The flexible indicating bands may indicate the characteristics of the contents of the container. In some embodiments, the plurality of bands may be removably attached to the container, and may be disposed around a circumference of the container.

In one or more embodiments, the indicating of information includes a fourth step S40 of enabling the user to identify the associated characteristics of the contents of the container by tactilely perceiving the plurality of protrusions on each of the plurality of flexible indicating bands without reliance on visual information. In some embodiments, a user may interact with the protrusions of the bands through touch (e.g., by a finger) to identify a quantity, shape, or the like, to identify a characteristic of the contents in the container, for example a dosage.

When a plurality of bands is attached to the container, each having a plurality of protrusions, a user may interact with the respective protrusions of each band to identify the tactile patterns (e.g., based on a shape, quantity, or the like) formed by the protrusions. The user may interact with protrusions of a first band of the plurality of bands to, based on the tactile patterns formed by the protrusions, identify a dosage of the contents of the container, and may interact with protrusions of a second band of the plurality of bands to, based on the tactile patterns formed by the protrusions, identify a type of the contents of the container.

In one or more embodiments, the indicating of information may include a fifth step of S50 of removing at least one of the plurality of bands from a first container and attaching the at least one of the plurality of bands to a second container (e.g., FIG. 4).

In one or more embodiments, the tactile patterns of the respective ones of the plurality of bands may represent a same characteristic for a plurality of containers. In some embodiments, a first band may represent a first characteristic corresponding to contents of a first container, and may represent a second characteristic corresponding to contents of a second container, the first and second characteristic being the same. In this way, when contents of different containers (e.g., differently shaped containers in FIG. 4) have a same characteristic associated therewith, the user may attach a same band to different containers.

In one or more embodiments, the indicating of information may include a fifth step of S50 of modifying the tactile pattern formed by the plurality of protrusions of the at least one of the plurality of bands to represent the different characteristic, the different characteristic being of contents of the second container. In some embodiments, the contents of the second container may have a characteristic different from that of the contents of the first container. In this case, a user may modify the tactile patterns formed by the protrusions of the band originally attached to the first container, to correspond to the characteristic of the contents of the second container. For example, if the contents of the second container have a different dosage, a user may remove or add a protrusion to change the quantity of protrusions on the band that may be interacted with by the user to represent the different dosage.

In one or more embodiments, different subsets or positions of the plurality of protrusions of at least one of the plurality bands represent different characteristics. In some embodiments, a first protrusion of the plurality of protrusions may have a first shape corresponding to a first characteristic, and a second protrusion of the plurality of protrusions may have a second shape corresponding to a second characteristic different from the first characteristic. In this way, a user may use the same band to indicate different types of information corresponding to the same container, for example, a dosage of the contents of the container and a type of the contents of the container.

In concluding the detailed description, those skilled in the art will appreciate that many variations and modifications can be made to the embodiments described herein without departing from the present disclosure. Therefore, the disclosed embodiments of the disclosure are to be understood in a generic and descriptive sense and not for purposes of limitation.

What is claimed is:

1. An indicating band configured to be removably attached to a plurality of containers having different external shapes, respectively, the indicating band comprising:
a body having a closed loop shape and comprising a flexible material; and
a protrusion on an outer surface of the body and extending away from the body.

2. The indicating band of claim 1, wherein the body comprises at least one of rubber, silicone, or polyurethane.

3. The indicating band of claim 1, wherein the protrusion is integrally formed with the body.

4. The indicating band of claim 1, wherein the protrusion is removably coupled to the body.

5. The indicating band of claim 4, wherein the protrusion is removably coupled to the body by at least one of a magnetic coupling, a screw coupling, or a hook-and-loop coupling.

6. The indicating band of claim 1, wherein the protrusion has at least one of a square shape, a rectangle shape, a triangle shape, a semi-triangle shape, semi-oval shape, or a semi-circular shape in a plan view.

7. The indicating band of claim 1, wherein the protrusion is a first protrusion,
wherein the indicating band further comprises a second protrusion on the outer surface of the body, extending away from the body, and spaced apart from the first protrusion, and
wherein the first protrusion has a first shape, and the second protrusion has a second shape that is different than the first shape.

8. The indicating band of claim 1, wherein the protrusion is a first protrusion,
wherein the indicating band further comprises a second protrusion on the outer surface of the body, extending away from the body, and spaced apart from the first protrusion, and
wherein the first protrusion has a first color, and the second protrusion has a second color that is different than the first color.

9. The indicating band of claim 1, wherein the protrusion has a width decreasing in a direction away from the body.

10. The indicating band of claim 1, wherein the protrusion has a height less than or equal to 3.0 millimeters.

11. The indicating band of claim 1, wherein the protrusion has a width less than or equal to a width of the body.

12. A system comprising:
a plurality of containers having different external shapes; and
a flexible indicating band configured to be individually, removably attached to respective ones of the plurality of containers, the indicating band comprising:
a body having a closed-loop shape and configured to individually extend around each of the plurality of containers; and
a plurality of protrusions on the outer surface of the body and extending away from the body, a first protrusion from among the plurality of protrusions having a shape different from that of a second protrusion from among the plurality of protrusions.

13. The system of claim 12, wherein the body is configured to extend around a first portion of at least one of the plurality of containers and around a second portion of the at least one of the plurality of containers, and
wherein the first and second portions of the at least one of the containers being spaced from each other and having different circumferences with respect to each other.

14. The system of claim 12, wherein the protrusion is removably coupled to the body.

15. The system of claim 12, wherein the first protrusion has a shape or color different from that of the second protrusion, the shape or the color corresponding to a dosage of contents of the at least one of the plurality of containers.

16. A method for indicating information associated with contents of a container to a user via tactile features on a flexible indicating band, the method comprising:
providing a plurality of flexible indicating bands, respective ones of the plurality of flexible indicating bands being configured to be removably attached around the container, and having a plurality of tactile protrusions;
associating one or more characteristics of the contents of the container with a tactile pattern formed by the plurality of tactile protrusions of the respective ones of the plurality of flexible indicating bands, the tactile patterns of the plurality of flexible indicating bands configured to be perceived by touch, and the characteristics comprising at least one of:
contents of the container;
a dosage or usage amount;
or a usage instruction;
attaching the plurality of flexible indicating bands to the container, the flexible indicating bands indicating the one or more characteristics of the contents of the container; and
enabling the user to identify the associated characteristics of the contents of the container by tactilely perceiving the plurality of protrusions on each of the plurality of flexible indicating bands without reliance on visual information.

17. The method of claim 16, wherein the tactile patterns of the respective ones of the plurality of flexible indicating bands represents a same characteristic for a plurality of containers.

18. The method of claim 16, wherein the method further comprises modifying the tactile patterns of the respective ones of the plurality of flexible indicating bands to represent a different characteristic.

19. The method of claim 18, wherein the method further comprises: removing at least one of the plurality of flexible indicating bands from a first container and attaching the at least one of the plurality of flexible indicating bands to a second container; and
modifying the tactile pattern formed by the plurality of protrusions of the at least one of the plurality of flexible indicating bands to represent the different characteristic, the different characteristic being of contents of the second container.

20. The method of claim 16, wherein different subsets or positions of the plurality of protrusions of at least one of the plurality of flexible indicating bands represent a different characteristic.
